(19) United States
(12) Patent Application Publication
(10) Pub. No.: US 2022/0176115 A1
(43) Pub. Date: Jun. 9, 2022

(54) ELECTROMAGNETIC DEVICE AND METHOD OF OPERATING THE SAME

(71) Applicant: Shan-Zu Lin, Taipei City (TW)

(72) Inventor: Shan-Zu Lin, Taipei City (TW)

(21) Appl. No.: 17/506,738

(22) Filed: Oct. 21, 2021

(30) Foreign Application Priority Data
Dec. 4, 2020 (TW) 109142922

(51) Int. Cl. A61N 1/36; H02J 50/20

(52) U.S. Cl. CPC A61N 1/3603; H02J 50/20; A61N 1/36021

(57) ABSTRACT

An electromagnetic device mating with a frequency specific microcurrent (FSM) to operate contains: a signal capture apparatus arranged on a wire between two current output ports of the FSM and including a frequency capture module. The frequency capture module is configured to determine a current output frequency of the FSM, and the current output frequency of the FSM is converted into a signal by the frequency capture module so as to send the signal. The signal is a specific controlling signal calculated from the current output frequency which corresponds to the frequency capture module.

### FIELD OF THE INVENTION

The present invention relates to an electromagnetic device mating with a frequency specific microcurrent (FSM) to operate and a method of operating the same, and more particularly to detect a current output frequency of a frequency specific microcurrent (FSM) in a wireless connection manner to produce different electromagnetic fields (MF) to restore the user auxiliary after resonance on the user.

### BACKGROUND OF THE INVENTION

At present, there are many kinds of electrotherapy devices in the market. Most principles of the electrotherapy devices are based on the electricity conducted to contact the skin of a user's body. By stimulating the skin, muscles and other human tissues, it produces a stimulation and relaxation. However, scientists discovered that different organs and tissues will produce “co-frequency effects”, such as fat, arteries, veins, bones, synovial sac, cartilage, intervertebral disc, dura mater, fascia, immune system, joint capsule, tendon, ligament, nerve, periosteum, skin, stomach, esophagus, large intestine, small intestine, liver, bladder, kidney, ureter, urethra, prostate, uterus, testicles, ovary, ureter, pituitary gland, adrenal gland, thyroid, parathyroid, pineal gland, lung, trachea, throat, cerebrum, cerebellum, midbrain, prolonged encephalon, spinal cord, sympathetic nerve, parasympathetic nerve and so on. If the frequency is further stimulated by microcurrent, the effect will be better. In short, the researchers found that the organs and tissues in the body will have a “specific frequency”, and if this frequency is found, it can produce a resonance effect with this organ and tissue, which will produce the auxiliary effect of adjusting and repairing specific portion of the user.

Referring to FIG. 1, a frequency specific microcurrent (FSM) 20 is sold by Inspirstar Inc, such as ┌IS02PRO Programmable Microcurrent Stimulator. The FSM 20 includes a control circuit in which multiple micro-current discharge programs of various frequencies are provided. The FSM 20 includes two current output ports connected with two wires L, and the two wires L are connected with multiple contact sheets P configured to contact with the user, such that the micro currents are delivered to a skin of the user via the two wires L and the multiple contact sheets P to cause a micro discharge E, and a control panel 21 is configured to set a power frequency mode, thus restoring the user.

Preferably, the FSM 20 is connected with a computer C via a transmission line S to download new information, thus updating a discharge program built in the FSM 20.

The present invention has arisen to mitigate and/or obviate the afore-described disadvantages.

### SUMMARY OF THE INVENTION

The primary aspect of the present invention is to provide an electromagnetic device which contains the signal capture apparatus configured to send the current output frequency of the FSM to the electromagnetic generator in the wireless connection manner or the wired connection manner so as to drive the magnetic field generator by using the specific controlling signal to produce different electromagnetic fields (MF) to restore the user auxiliary after resonance on the user.

To obtain above-mentioned aspect, an electromagnetic device provided by the present invention contains: a signal capture apparatus including arranged on a wire between two current output ports of the FSM (and including a frequency capture module. The frequency capture module is configured to determine a current output frequency of the FSM, and the current output frequency of the FSM is converted into a signal by the frequency capture module so as to send the signal, wherein the signal is a specific controlling signal calculated from the current output frequency which corresponds to the frequency capture module.

A method of operating an electromagnetic device comprises steps of:

A) capturing, wherein two current output ports of a FSM are connected with a signal capture apparatus, and the signal capture apparatus includes a frequency capture module, the frequency capture module is configured to determine a current output frequency of the FSM, and the current output frequency of the FSM is converted into a wireless signal by the frequency capture module so as to send the wireless signal;

B) driving, wherein an electromagnetic generator contacts with a skin of a user, and the electromagnetic generator includes a Bluetooth frequency calculation module, a gate integrated circuit (IC), and a magnetic field generator, wherein the Bluetooth frequency calculation module is configured to receive a wireless signal of the frequency capture module and to mate with the current output frequency of the FSM so as to output an electromagnetic control signal to the gate IC, wherein the gate IC is configured to receive the electromagnetic control signal from the Bluetooth frequency calculation module and to send a specific controlling signal to the magnetic field generator; and

C) restoring, wherein the magnetic field generator is driven by the specific controlling signal of the gate IC to produce different electromagnetic fields (MF), thus restoring the user auxiliary.

### BRIEF DESCRIPTION OF THE DRAWINGS

FIG. 1 is a schematic view of a conventional electromagnetic device.

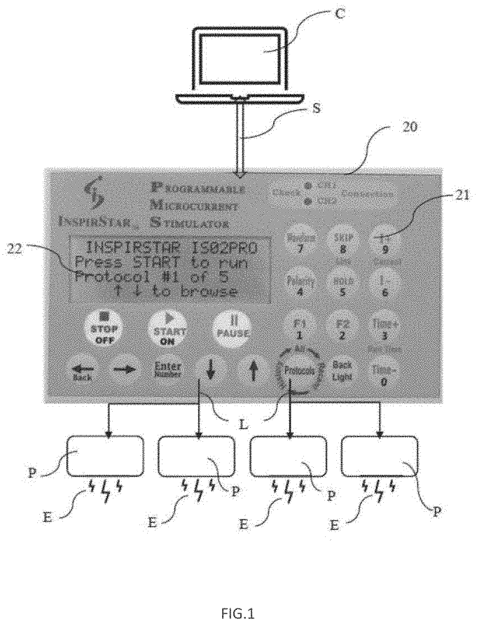

FIG. 2 is a schematic view showing the assembly of an electromagnetic device according to a first embodiment of the present invention.

FIG. 3 is another schematic view showing the assembly of a part of the electromagnetic device according to the first embodiment of the present invention.

FIG. 4 is a schematic view showing the assembly of a part of an electromagnetic device according to a second embodiment of the present invention.

FIG. 5 is a schematic view showing the assembly of a part of an electromagnetic device according to a third embodiment of the present invention.

### DETAILED DESCRIPTION OF THE PREFERRED EMBODIMENTS

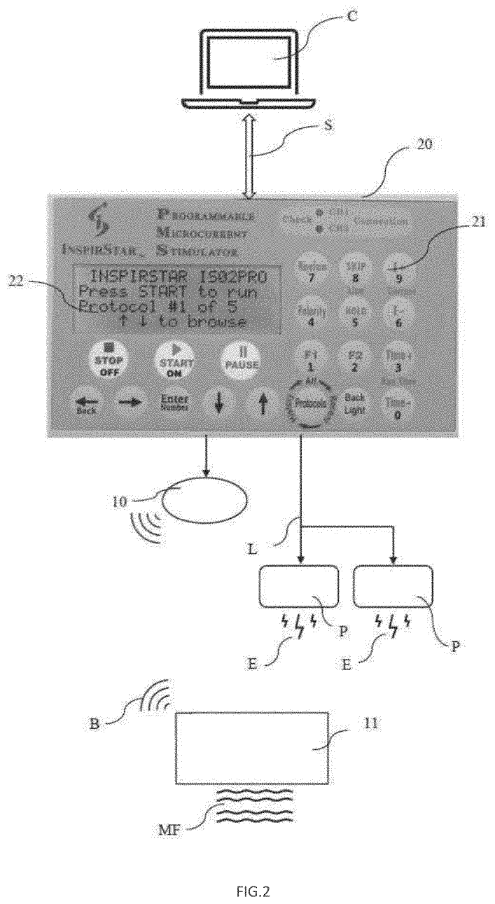

With reference to FIG. 2, an electromagnetic device according to a first embodiment of the present invention matches with a frequency specific microcurrent (FSM) 20 so as to output micro currents and to supply resonance to a user, the electromagnetic device is arranged on a wire L between two current output ports of the FSM 20 and includes a signal capture apparatus 10 and an electromagnetic generator 11, wherein the signal capture apparatus 10 is configured to capture micro-current frequency from the FSM 20 and to send a micro-current frequency signal to the electromagnetic generator 11 via a wireless signal. The micro-current frequency signal is sent via the wireless signal by using Bluetooth system, and the signal capture apparatus 10 captures the micro-current frequency from the FSM 20 and converts the micro-current frequency into a wired signal outputted by a signal cable.

The wire of the FSM 20 has multiple contact sheets P extending therefrom and configured to contact with the user, such that the micro currents are sent to a skin of the user via the wire L and the multiple contact sheets P from the FSM 20 so as to cause a micro discharge E, and a control panel 21 is configured to set a power generation frequency module, thus adjustably restoring a specific portion of the user.

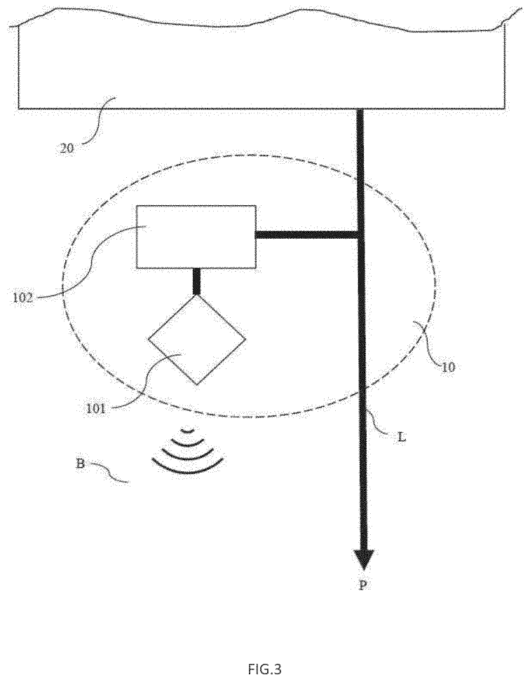

Referring to FIG. 3, an end of the signal capture apparatus 10 is electrically connected with the two current output ports of the FSM 20, and the signal capture apparatus 10 includes an electric circuit on which a frequency capture module 101 is connected and is configured to determine a current output frequency of the FSM 20, and the current output frequency of the FSM 20 is converted into a wireless signal B by the frequency capture module 101 so as to send the wireless signal B to the electromagnetic generator 11. The frequency capture module 101 is connected with a first power supply portion 102 configured to supply a power to at least one battery/cell directly or via an external power supply, such that when the frequency capture module 101 is connected with the FSM 20, the frequency capture module 101 determines the current output frequency of the FSM 20 and does not influence a current output of the FSM 20, thus maintaining a micro-current discharge frequency of FSM 20. The frequency capture module 101 or the wire L is connected with two current output ports of the FSM 20 alternatively.

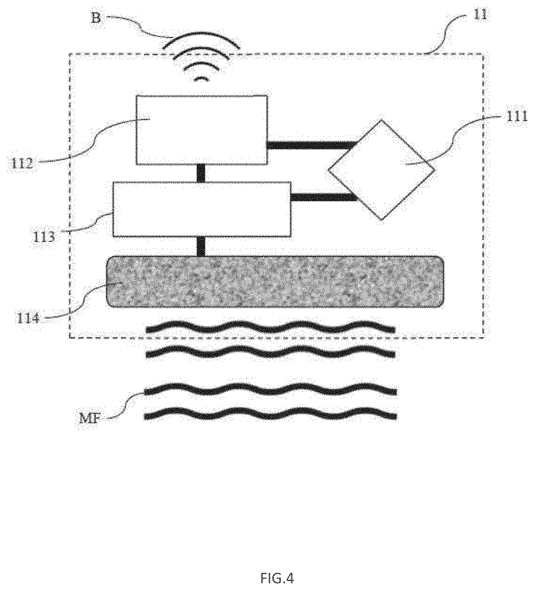

As shown in FIG. 4, in a second embodiment, the electromagnetic generator 11 contacts with the skin of the user with the multiple contact sheets P, and the electromagnetic generator 11 includes a second power portion 111, a Bluetooth frequency calculation module 112, a gate integrated circuit (IC) 113, and a magnetic field generator 114, wherein the second power portion 111 is configured to supply the power via the at least one battery/cell or the external power supply and is electrically connected with the Bluetooth frequency calculation module 112 and the gate IC 113, wherein the Bluetooth frequency calculation module 112 is configured to receive the wireless signal B of the frequency capture module 101 and to mate with the current output frequency of the FSM 20, thus outputting an electromagnetic control signal to the gate IC 113. The gate IC 113 is electrically connected with the magnetic field generator 114 and is configured to receive the electromagnetic control signal from the Bluetooth frequency calculation module 112 and to send a specific controlling signal to the magnetic field generator 114 so that the magnetic field generator 114 is driven by the specific controlling signal to produce different electromagnetic fields (MF). In another embodiment, the magnetic field generator 114 receives the current output frequency of the FSM 20 in a wired connection manner and outputs the electromagnetic control signal to the gate IC 113.

In the first embodiment, a current generator 12 outputs the micro currents to the skin of the user via the wire L and the multiple contact sheets P from the FSM 20 so as to cause the micro discharge E.

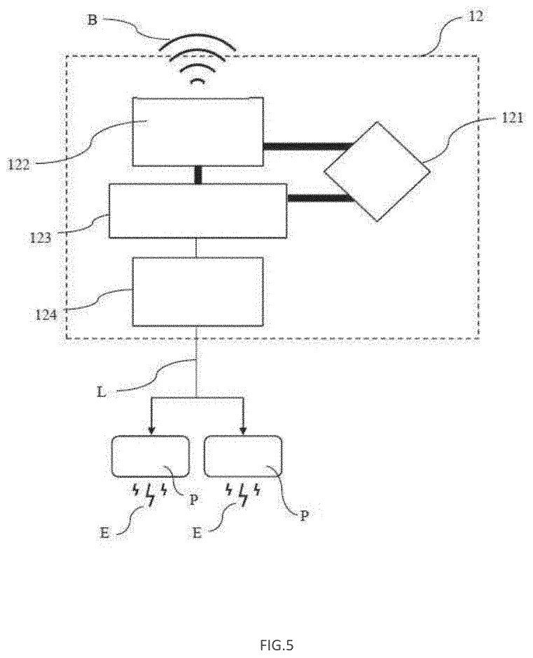

Referring to FIG. 5, in a third embodiment, a second power portion 121, a Bluetooth frequency calculation module 122, a gate IC 123, and a micro current generator 124 are connected with a wire L, and the wire L has multiple contact sheets P configured to contact with the user and to cause a micro discharge E to the skin of the user, wherein the second power portion 121 is configured to supply the power via the at least one battery/cell or the external power supply and is electrically connected with the Bluetooth frequency calculation module 122 and the gate IC) 123, wherein the Bluetooth frequency calculation module 122 is configured to receive the wireless signal B of the frequency capture module 101 and to mate with the current output frequency of the FSM 20, thus outputting an electromagnetic control signal to the gate IC 123. The gate IC 123 is electrically connected with the micro current generator 124 and is configured to receive the electromagnetic control signal from the Bluetooth frequency calculation module 122 and to send a specific controlling signal to the micro current generator 124 so that the micro current generator 124 is driven by the specific controlling signal to output the micro currents to the multiple contact sheets P via the wire L, thus causing the micro discharge E to the skin of the user via the multiple contact sheets P. The current generator 12 extends the current output frequency of the FSM 20 in a wired connection manner so as to output the electromagnetic control signal.

The signal capture apparatus 10 sends the current output frequency of the FSM 20 to the electromagnetic generator 11 in the wireless connection manner or the wired connection manner so as to drive the magnetic field generator 114 by using the specific controlling signal to produce different electromagnetic fields (MF), thus restore the user auxiliary after resonance on the user.

In another application, the electromagnetic generator 11 is solely put close to the skin of the user, wherein when the FSM 20 does not use the multiple contact sheets P, the current output frequency of the FSM 20 is sent to the electromagnetic generator 11 so that the magnetic field generator 114 is driven by the specific controlling signal to produce different electromagnetic fields (MF), thus restoring the user auxiliary.

A method of operating an electromagnetic device comprises steps of:

A) capturing, wherein two current output ports of a FSM 20 are connected with a signal capture apparatus 10, the signal capture apparatus 10 is connected with a frequency capture module 101 and is configured to determine a current output frequency of the FSM 20, and the current output frequency of the FSM 20 is converted into a wireless signal B by the frequency capture module 101 so as to send the wireless signal B to the electromagnetic generator 11, wherein when the frequency capture module 101 is connected with the FSM 20, the current output frequency of the FSM 20 is not influenced by the frequency capture module 101, thus maintaining a micro-current discharge frequency of FSM 20;

B) driving, wherein an electromagnetic generator 11 contacts with a skin of a user, and the electromagnetic generator 11 includes a Bluetooth frequency calculation module 112, a gate integrated circuit (IC) 113, and a magnetic field generator 114, wherein the Bluetooth frequency calculation module 112 is configured to receive a wireless signal B of the frequency capture module 101 and to mate with the current output frequency of the FSM 20 so as to output an electromagnetic control signal to the gate IC 113, wherein the gate IC 113 is configured to receive the electromagnetic control signal from the Bluetooth frequency calculation module 112 and to send a specific controlling signal to the magnetic field generator 114 so that the magnetic field generator 114 is driven by the specific controlling signal; and

C) restoring, wherein the magnetic field generator 114 is driven by the specific controlling signal of the gate IC 113 to produce different electromagnetic fields (MF), thus restoring the user auxiliary.

While the preferred embodiments of the invention have been set forth for the purpose of disclosure, modifications of the disclosed embodiments of the invention as well as other embodiments thereof may occur to those skilled in the art. The scope of the claims should not be limited by the preferred embodiments set forth in the examples, but should be given the broadest interpretation consistent with the description as a whole.

What is claimed is:

1. An electromagnetic device mating with a frequency specific microcurrent (FSM) to operate comprising:
a signal capture apparatus (10) including arranged on a wire (L) between two current output ports of the FSM (20) and including a frequency capture module (101), the frequency capture module (101) being configured to determine a current output frequency of the FSM (20), and the current output frequency of the FSM (20) is converted into a signal by the frequency capture module (101) so as to send the signal, wherein the signal is a specific controlling signal calculated from the current output frequency which corresponds to the frequency capture module (101).

2. The electromagnetic device as claimed in claim 1, wherein the frequency capture module (101) determines the current output frequency of the FSM (20) and does not influence a current output of the FSM (20), thus maintaining a micro-current discharge frequency of the FSM (20).

3. The electromagnetic device as claimed in claim 1, wherein the frequency capture module (101) is connected with a first power supply portion (102).

4. The electromagnetic device as claimed in claim 1, wherein the frequency capture module (101) determines the current output frequency of the FSM (20), the current output frequency of the FSM (20) is converted into a wireless signal (B) by the frequency capture module (101) so as to send the wireless signal (B), wherein an electromagnetic generator (11) includes a Bluetooth frequency calculation module (112), a gate integrated circuit (IC) (113), and a magnetic field generator (114), wherein the Bluetooth frequency calculation module (112) is configured to receive a wireless signal (B) of the frequency capture module (101) and to output an electromagnetic control signal to the gate IC (113), the gate IC (113) is configured to receive the electromagnetic control signal from the Bluetooth frequency calculation module (112) so that the magnetic field generator (114) is driven to produce different electromagnetic fields (MF).

5. The electromagnetic device as claimed in claim 4, wherein the electromagnetic generator (11) includes a second power portion (111), and the second power portion (111) is electrically connected with the Bluetooth frequency calculation module (112) and the gate IC (113).

6. An electromagnetic device mating with a frequency specific microcurrent (FSM) to operate comprising: an electromagnetic generator (11) including a gate integrated circuit (IC) (113) and a magnetic field generator (114), wherein the gate IC (113) is configured to receive the electromagnetic control signal so as to drive the magnetic field generator (114) to produce different electromagnetic fields (MF).

7. The electromagnetic device as claimed in claim 6, wherein the electromagnetic generator (11) further includes a Bluetooth frequency calculation module (112), and the Bluetooth frequency calculation module (112) is configured to receive a signal of the frequency capture module (101).

8. The electromagnetic device as claimed in claim 6, wherein the electromagnetic generator (11) includes a second power portion (111).

9. An electromagnetic device mating with a frequency specific microcurrent (FSM) to operate comprising: a current generator (12), and the current generator (12) including a gate integrated circuit (IC) (123) and a micro current generator (124) which are connected with a wire (L), and the wire (L) has multiple contact sheets (P) configured to contact with a user and to cause a micro discharge (E) to a skin of the user, wherein the gate IC (123) is electrically connected with the micro current generator (124) and is configured to receive the electromagnetic control signal and to drive the micro current generator (124) to output micro currents to the multiple contact sheets (P) via the wire (L), thus causing the micro discharge (E) to the skin of the user via the multiple contact sheets (P).

10. The electromagnetic device as claimed in claim 9, wherein current generator (12) further includes a Bluetooth frequency calculation module (122) which is configured to receive the signal of the frequency capture module (101).

11. The electromagnetic device as claimed in claim 9, wherein the current generator (12) further includes a second power portion (121).

12. A method of operating an electromagnetic device comprises steps of:
A) capturing, wherein two current output ports of a FSM (20) are connected with a signal capture apparatus (10), and the signal capture apparatus (10) includes a frequency capture module (101), the frequency capture module (101) is configured to determine a current output frequency of the FSM (20), and the current output frequency of the FSM (20) is converted into a wireless signal (B) by the frequency capture module (101) so as to send the wireless signal (B);
B) driving, wherein an electromagnetic generator (11) contacts with a skin of a user, and the electromagnetic generator (11) includes a Bluetooth frequency calculation module (112), a gate integrated circuit (IC) (113), and a magnetic field generator (114), wherein the Bluetooth frequency calculation module (112) is configured to receive a wireless signal (B) of the frequency capture module (101) and to mate with the current output frequency of the FSM (20) so as to output an electromagnetic control signal to the gate IC (113), wherein the gate IC (113) is configured to receive the electromagnetic control signal from the Bluetooth frequency calculation module (112) and to send a specific controlling signal to the magnetic field generator (114); and
C) restoring, wherein the magnetic field generator (114) is driven by the specific controlling signal of the gate IC (113) to produce different electromagnetic fields (MF), thus restoring the user auxiliary.

13. The electromagnetic device as claimed in claim 12, wherein the frequency capture module (101) determines the current output frequency of the FSM (20) and does not influence a current output of the FSM (20), thus maintaining a micro-current discharge frequency of the FSM (20).

14. The electromagnetic device as claimed in claim 12, wherein the frequency capture module (101) is connected with a first power supply portion (102).

15. The electromagnetic device as claimed in claim 12, wherein the Bluetooth frequency calculation module (112) and the gate IC (113) is electrically connected with a second power portion (111).
